(19) United States
(12) Patent Application Publication
(10) Pub. No.: US 2013/0190894 A1
(43) Pub. Date: Jul. 25, 2013

(54) LEG PROSTHESIS

(71) Applicant: Horst Gingel, Dreisen (DE)

(72) Inventor: Horst Gingel, Dreisen (DE)

(21) Appl. No.: 13/817,555

(22) PCT Filed: Aug. 18, 2011
(86) PCT No.: PCT/EP2011/064219

§ 371 Date: Apr. 11, 2013

(30) Foreign Application Priority Data
Aug. 19, 2010 (DE) 10 2010 034 880.5

(51) Int. Cl. A61F 2/80

(52) U.S. Cl. CPC A61F 2/80
USPC 623/33

(57) ABSTRACT

A leg prosthesis, including an inner tube having a longitudinal axis a and an outer tube which is mounted on the inner tube and can be displaced relative to the inner tube in the axial direction relative to the longitudinal axis a, a retaining device being provided, by means of which the inner tube and the outer tube can be positively secured relative to one another in the axial direction relative to the longitudinal axis a in at least one position L. The retaining device includes an operating element which can be manipulated by the prosthesis wearer and by which the retaining device can be detached such that, proceeding from the position L, the inner tube can be continuously displaced in the axial direction relative to the longitudinal axis a relative to the outer tube into at least one position K, whereby the leg prosthesis can be shortened and, by the application of a force F which acts on the inner tube and/or on the outer tube and acts in the axial direction, the inner tube can be displaced, starting from the position K, in the axiai direction relative to the longitudinal axis a relative to the outer tube, whereby the leg prosthesis can be continuously extended, it being possible for the inner tube and the outer tube to be automatically secured in the axial direction relative to the longitudinal axis a only in the position L by the retaining device.

### FIELD OF THE INVENTION

The invention relates to a ieg prosthesis, comprising an outer tube having a longitudinal axis a and an inner tube able to move relative to the outer tube in the direction of the longitudinal axis. A iockable retaining means is provided, by which the inner tube can be positively locked in the direction of the longitudinal axis a relative to the outer tube in at least one position L.

### BACKGROUND OF THE INVENTION

There is already known a length-adjustable leg prosthesis from DE 328 107 A, in which the free end of a spring fastened to the shin engages by means of pins in a lock piece, connected to the knee joint, under the pressure of a pressing screw working against the spring action. In order to adjust the length to the wearer of the prosthesis, the screw can be loosened by means of a tool, the spring pins are disengaged, the length of the prosthesis is adjusted to one of the predetermined measures within the locking element and the screw is tightened once again after Inserting the spring pin.

DE 600 18 987 T2 describes a pneumatically or hydraulically adjustable teg prosthesis with a dual piston, in which two parts of the leg prosthesis can move in the axial direction. For the transfer of the medium from one side of the piston to the other, a valve is provided, which is operated manually by the prosthesis wearer or electrically, by which the prosthesis wearer can change the length setting. The change of length is possibly by flow transfer of the medium. The original position of the two parts of the leg prosthesis is not mechanically defined here, so that a resetting of the length in the original position can only be done by how the prosthesis wearer feels it to be.

Basically the problem with leg and especially lower leg prostheses is that when seated, i.e., with the joint bent, the shaft receiving the leg stump presses on the stump. To counter this pressure, the prosthesis wearer is forced to stretch their leg somewhat, but this is not always possible in the case of larger persons, due to space considerations, especially in a car or airplane. Furthermore, the lower leg prosthesis is taller than the lower leg which it is replacing, so that when sitting the prosthesis extends beyond the intact lower leg.

### SUMMARY OF THE INVENTION

The problem of the invention is to configure and arrange, such a leg prosthesis so that a temporary shortening of the length is possible by easy manipulation of the prosthesis wearer, and so is an automatic return to the starting position.

The problem is solved according to the invention in that the retaining means has a manually movable operating element, by which the retaining means is movable, so that one tube can be loosened in position L relative to the other tube and can be moved continuously by a force F info a position K, defined by an end stop with positive action in the direction of the longitudinal axis a. Furthermore, the one tube starting from position K or any given position X between position K and position L can be moved automatically in the direction of the longitudinal axis a into position L solely by the force of gravity G acting on the tube, and in the exact position L the two tubes bear against each other by the positive end stop acting in the direction of the longitudinal axis a and are automatically secured by the retaining means.

Thus, the leg prosthesis can be moved info two positions. The “long” position L for the active wearing of the leg prosthesis, in which the prosthesis wearer can stand and run. In this position Lthe leg prosthesis is stiff, it neither changes in length nor twists around. This position is individually adjusted precisely to the prosthesis wearer by the orthopedist. Starting from position L, the leg prosthesis according to the invention can be loosened by a simple motion at the retaining means, so that the two tubes move relative to each other and the leg prosthesis is shortened. In this “short” position K, the length I of the leg prosthesis is defined by an end stop, at which the two tubes bear against each other in the direction of the longitudinal axis a. This position K is achieved after the loosening of the retaining means, because the leg moves downward and the two tubes are shoved one into the other. In this unstable position K, the two tubes do not lock together. Therefore, as soon as the prosthesis wearer lifts his leg again, the one tube slips out from the other tube by exactly the same dimension that it was previously shoved into it. Thus, the position K can be maintained only as long as the leg prosthesis is at least slightly loaded. This loading is guaranteed in particular when the prosthesis wearer is sitting.

But once the prosthesis wearer lifts his leg so that the two tubes are again moved apart into the exact position L, the two tubes are automatically secured to one another again. Thus, if the prosthesis wearer stands up and lifts his leg, the two tubes solely by the force of gravity again slide apart and are automatically secured, without the prosthesis wearer having to do anything. The leg can again be fully loaded in position L for the purpose of standing and walking.

The process of shortening or lengthening the leg prosthesis can be performed as often as one likes within a few seconds. The shortening is done preferably while seated. The lengthening can also be done while sitting or standing.

The movement from one of the two positions into the other occurs continuously. The degree of shortening is dictated by the leg prosthesis and cannot be individually adjusted by the prosthesis wearer. However, the degree of shortening can be adjusted individually to the prosthesis wearer during the fabrication of the leg prosthesis.

In this way, the prosthesis wearer can shorten the leg prosthesis for sitting down, so that the angle between thigh and shin can be increased and the load on the shaft can be eased. Furthermore, both shins have the same height while seated.

For this, it is preferable that the outer tube can be secured by the retaining means to the inner tube only in the position L defined by the positive-action end stop. While basically different rigid intermediate positions are conceivable, which the prosthesis wearer could set as an alternative to position L or position K, a free and continuous variability increases the wearing comfort, since with, the already described single operation and single loosening no further manipulation steps are needed in order to sit in a single correct position K and stand or walk in a single correct position L. Alternative positions L are not relevant for standing or walking, and for sitting the greatest possible shortening to the position K affords the most wearing comfort.

For this it can also be advantageous for the leg prosthesis to be shortened by a dimension M of at least 5 cm, starting from the position L, by the movement of the two tubes, and to be lengthened by the same dimension M of at least 5 cm, starting from the position K. Depending on the overall length of the leg prosthesis, adjustment dimensions greater than 10 cm can also be achieved. The dimension depends on the geometry of the leg prosthesis and the individual adjustments to the prosthesis wearer.

Moreover, it can be advantageous to provide, as the retaining means, a spring-loaded locking bolt that can move in the radial direction to the longitudinal axis a, being mounted by a bolt housing on the outer tube and able to lock inside a borehole in the inner tube or in a hardened bushing in the inner tube. The locking bolt is a simple, safe, and permanently very reliable retaining means, which can be combined in easy manner with an easily manipulated operating element. Due to space considerations and the geometry of the two tubes, a locking bolt that acts in the radial direction is very easily integrated into the whole. The spring-loaded bolt is flush in position L with a cavity or borehole, in which it locks automatically for securing purposes. The free play between bolt and borehole should be such that the leg prosthesis is sufficiently rigid in the direction of the longitudinal axis a.

For the locking bolt to jump easily into the borehole, the borehole must be somewhat larger than the locking bolt itself. But this results in a slackness between these two transmission elements. If a separate end stop were provided for position L, position L would be defined by this end stop and a corresponding end stop position could be achieved very precisely. Then the locking bolt could also Sock inside the borehole with less slackness.

In another preferred embodiment, the locking bolt and the borehole are conical in shape, which allows for more slackness in position L and at the same time enables a tight locking in the direction of the tubes.

The prosthesis wearer can loosen the locking bolt at its knob, easily and without great effort, in order to shorten the leg prosthesis. The locking bolt can also be easily operated underneath pants, so that the steps needed to shorten the leg prosthesis can be done by a short hand movement.

Basically, the locking bolt can also be movable tangentially to the tubes, when this is advantageous. Moreover, alternative retaining means such as a locking washer with groove or a split pin.

It can also be advantageous to configure the locking bolt so that the locking in position L can be perceived acoustically and/or tactilely by the wearer of the leg prosthesis. When the spring-loaded locking bolt drives into the borehole with sufficient speed, a sufficiently large momentum is created, which is distinctly audible and perceptible to the prosthesis wearer. It is also conceivable for the bolt in the locked state inside the borehole to interact with a position indicator, such as a spring-loaded stylus, whose position the prosthesis wearer can feel in the locked state.

In connection with the configuration and arrangement according to the invention it can be beneficial for the inner tube to be able to move in the axial direction to the longitudinal axis by a plain bearing in the outer tube. The bearing enables a coaxial guidance of the two tubes and, depending on the choice of material for the bearing, a noiseless movement of the two tubes.

For this, it can be advantageous for the outer tube and the inner tube to each be configured as a tube slit in the direction of the longitudinal axis a or a closed tube. A closed tube will be used most often in the construction of such leg prostheses, due to the manufacturing advantages.

In terms of the stability of the leg prosthesis, it is advantageous to provide a guide pin on the outer tube and a groove on the inner tube, where the guide pin led in the groove forms a tongue and groove connection, which secures the two tubes against twisting in the circumferential direction to the longitudinal axis a. Typical leg prostheses are made from an individually adapted piece of pipe, which is naturally stiff to torsion. The length adjustability and the two tubes necessary for this, according to the invention, must be secured against torsion. A preferred connection is the familiar tongue and groove connection, which is appropriately adapted and also used here. In the preferred embodiment, the tongue is fashioned as a bolt, because the bolt in the form of a worm screw can be screwed afterwards into the groove after the two tubes are assembled together.

It is of special importance for the present invention that the retaining means or the guide pin or the outer tube are fashioned as end stop means that can bear against the inner tube in the position L or in the position K. Thus, the position K can be adjusted in the easiest way and be held without problem by the load of the leg.

### BRIEF DESCRIPTION OF THE DRAWINGS

Further benefits and details of the invention, are explained in the patent claims and in the specification and represented in the figures. There are shown:

FIG. 1a, a perspective representation of a leg prosthesis with an outer tube and art inner tube in position L;

FIG. 1b, a perspective representation of a leg prosthesis according to FIG. 1a in the shortened position K;

FIG. 2, a cross sectional view of the outer tube of the leg prosthesis along A-A′ of FIG. 3a;

FIG. 3a, a cross sectional view of the leg prosthesis in position L;

FIG. 3b, a cross sectional view of the leg prosthesis according to FIG. 3a in position K.

### DETAILED DESCRIPTION OF THE INVENTION

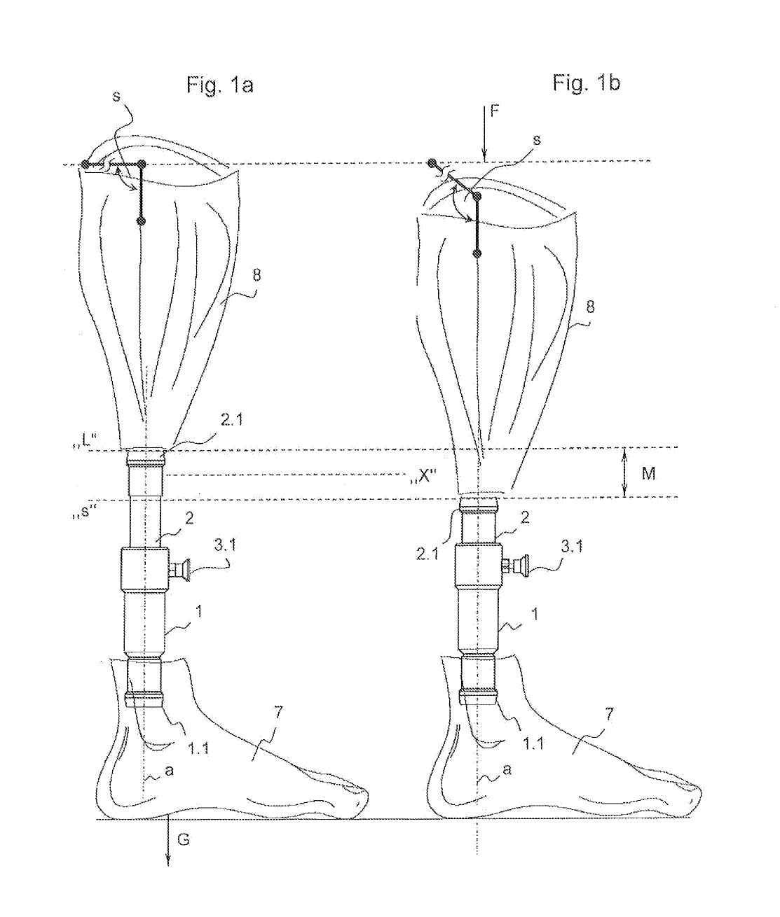

A leg prosthesis, as is shown in FIG. 1a to 3b, has two telescopic tubes 1, 2, an outer tube 1 and an inner tube 2 that is guided in the outer tube 1. At the lower end of the outer tube 1 is arranged an adapter 1.1 which serves to secure a foot fitting part 7. A similar adapter 2.1 is arranged on the inner tube 2, where a shaft 8 is secured (FIG. 1a and 1b). The leg prosthesis can be moved into two positions, L and K.

In position L, the leg prosthesis is longer than in position K and also rigid, so that like a one-piece pipe it cannot be moved either in length 1 or in a direction about the longitudinal axis a. This position L serves the designed use for standing and walking of the prosthesis wearer.

Starting from this position L, the leg prosthesis can be shortened in a position K, as is shown in FIG. 1b. In this position K, the two tubes 1, 2 bear against each other in the direction of the longitudinal axis a across corresponding end stop means 6, 6.1. The end stop is reached by moving the shaft 8 downward in the direction of the foot fitting part 7. This position allows the prosthesis wearer to sit and affords a larger angle s between the thigh and shin as compared to position L in this way, there is less load on the leg stump, which sits in the shaft 8.

By manual operation of the retaining means 3 in the form of a locking bolt 3, the two tubes 1, 2 can be loosened, starting from the position L, with a single hand movement. The load acting on the inner tube 2, situated on top, creates a force F, which lies within the discretion of the prosthesis wearer, so that the two tubes 1, 2 automatically and continuously slide one into the other and the leg prosthesis is shortened by the maximum dimension M in position K, In this position K, the two tubes 1, 2 are not secured to each other in the direction of the longitudinal axis a, so that there can be as many intermediate positions X as desired between position K and position L in which the two tubes 1, 2 could be moved relative to each other in the direction of the longitudinal axis a.

Starting from position K, the prosthesis wearer lifts his leg by at least the dimension M, so that the lower part of the leg prosthesis is automatically moved by force of gravity G back to the exact position L and the retaining means 3 engages, so that the leg prosthesis is again rigid in the direction of the longitudinal axis a. Even if the leg were lifted again by the dimension M, the two tubes 1, 2 would likewise automatically lock relative to each other in position L.

As is shown in FIGS. 3a and 3b, the two tubes 1, 2 are secured by the retaining means 3 in the form of a locking bolt 3, which engages directly in a borehole 2.3 (FIG. 3b) or in a hardened bushing 2.4 inserted in the borehole 2.3 (FIG. 3a). The two tubes 1, 2 can move relative to each other when the retaining means 3 is taken out from the borehole 2.3 or the bushing 2.4. Thus, the two tubes 1, 2 are mechanically and positively coupled to each other in position L. The inner tube 2 is configured as an inner pipe and can be guided or can travel axially in the outer tube 1, configured as an outer pipe, in the axial direction to the longitudinal axis a.

The outer pipe 1 has an oblong hole 2.2, which runs parallel to the longitudinal axis a, while inside the oblong hole 2.2 is arranged the tongue of the outer pipe 1, fashioned as a guide pin 6.1. The tongue and groove connection 6 so formed prevents a twisting of the two tubes 1, 2 about the longitudinal axis a.

Moreover, the tongue and groove connection 6 per FIG. 3b forms an end stop between outer pipe 1 and inner pipe 2 both in position L and in position K. The guide pin 6.1 here forms an end stop means that bears against the oblong hole 2.2 each time in both positions L and K in the direction of the longitudinal axis a, as is shown in FIGS. 3a and 3b, The length 1 of the oblong hole 2.2 thus corresponds to the dimension M.

In one sample embodiment not shown, the outer pipe 1 and the adapter 1.1 lie against each other in position K, so that the two end faces form end stop means in the direction of the longitudinal axis a.

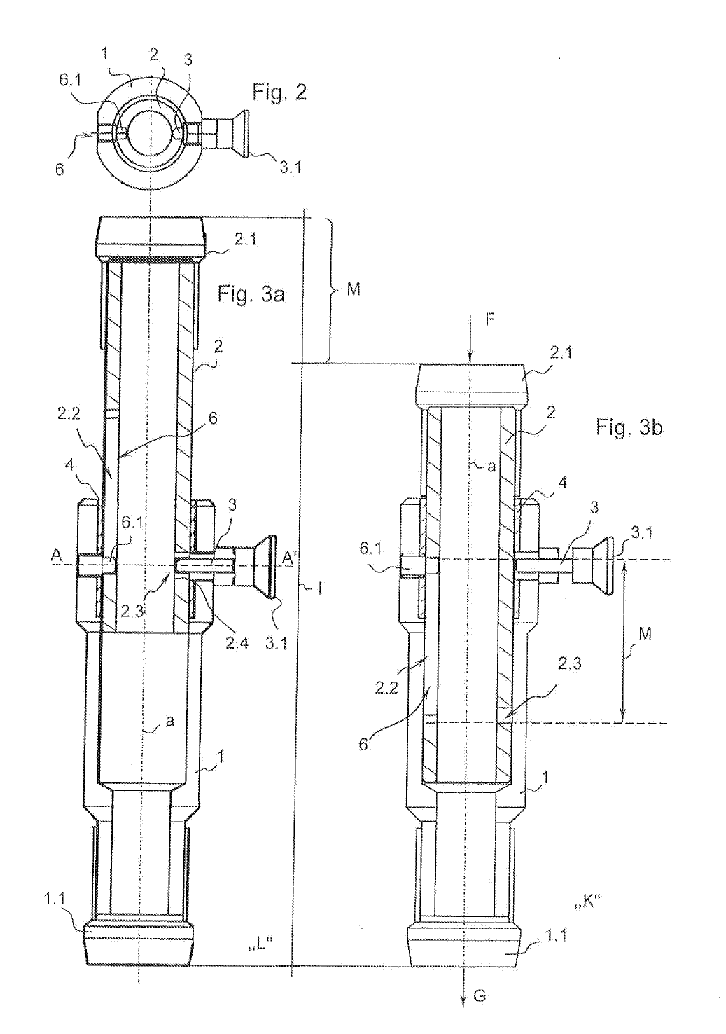

In FIGS. 2 to 3b, the retaining means fashioned as a locking bolt 3 is shown more closely, and it can be manually operated by the prosthesis wearer using an operating element 3.1 to loosen the locked connection. The locking bolt 3 in position L is arranged inside the borehole 2.3 or inside the bushing 2.4 in order to form a positive locking, blocking the axial displacement relative to the longitudinal axis a, so that a relative movement in the direction of the longitudinal axis a between the inner pipe 2 and the outer pipe 1 is prevented by the positive locking.

Starting from position L shown in FIG. 3a, the leg prosthesis can be shortened per FIG. 3b. For this, the locking bolt 3 is manually pulled radially outward from the bushing 2.4 installed in the borehole 2.3, preferably being spring-loaded, by means of the operating element 3.1, so that the inner pipe 2 can move coaxially inside the outer pipe 1, fashioned as a cylinder, in the downward direction here.

In order to ensure this displacement movement coaxially to the longitudinal axis a, the outer pipe 1 has a plain bearing 4 or a plain bearing bushing, inside which the inner pipe 2 is led, being fashioned as a piston.

In this position K reached by the two tubes 1, 2, no locking by the locking bolt 3 occurs. The locking bolt 3 is spring-loaded and bears against the outer surface of the inner pipe 2 and ensures a continuous displacement of the two tubes 1, 2, or the inner pipe 2 inside the outer pipe 1, downward starting from position L per FIG. 3a, by the force F, and upward starting from position K per FIG. 3b, by the force of gravity G.

Only when the completely extended exact position L of the two tubes 1, 2 is reached, i.e., a movement of the inner pipe 2 as far as the lower end stop, formed by the tongue and groove connection 6, in the oblong hole 2.2 in the inner pipe 2, does a locking occur by the locking bolt 3 by virtue of automatic engaging in the bushing 2.4 in the borehole 2.3, namely, a positive locking between the two tubes 1, 2, operating in the axial direction.

Thanks to a spring loading of the locking bolt 3, not shown, upon reaching the position L in which the locking bolt 3 and the corresponding borehole 2.3 or bushing 2.4 are flush in the axial direction, the bolt automatically moves into the borehole 2.3 and brings about the locked position.

In FIG. 2, the positions of the locking bolt 3 and the guide pin 6.1 in the radial direction to the longitudinal axis a are shown. The locking bolt 3 protrudes fully into the borehole 2.3, and the guide pin 6.1 likewise protrudes fully into the oblong hole 2.2.

What is claimed is:

1. A leg prosthesis, comprising: an outer tube having a longitudinal axis a and an inner tube able to move relative to the outer tube in the direction of the longitudinal axis, wherein a lockabie retaining means is provided, by which the inner tube can be positively locked in the direction of the longitudinal axis a relative to the outer tube in at least one position L,
wherein a) the retaining means has a manually movable operating element, by which the retaining means is movable, so that one tube can be loosened in position L relative to the other tube and can be moved continuously by a force F into a position K, defined by an end stop with positive action in the direction of the longitudinal axis a, and
wherein b) the one tube starting from position K or any given position X between position K and position L can be moved automatically in the direction of the longitudinal axis a into position L solely by the force of gravity G acting on the tube, and in the exact position L the two tubes bear against each other by the positive end stop acting in the direction of the longitudinal axis a and are automatically secured by the retaining means.

2. The leg prosthesis according to claim 1, wherein the outer tube can be secured by the retaining means to the inner tube only in the position L defined by the positive-action end stop.

3. The leg prosthesis according to claim 1, wherein the leg prosthesis can be shortened by a dimension M of at least 5 cm, starting from the position L, by the movement of the two tubes, and lengthened by the same dimension M of at least 5 cm, starting from the position K.

4. The leg prosthesis according to claim 1, wherein as the retaining means, a spring-loaded locking bolt is provided that can move in a radial direction to the longitudinal axis a, being mounted directly or indirectly on the outer tube and able to lock inside a borehole in the inner tube or in a hardened bushing in the inner tube.

5. The leg prosthesis according to claim 4, wherein the locking bolt is configured so that the locking in position L can be perceived acoustically and/or tactilely by a wearer of the leg prosthesis.

6. The leg prosthesis according to claim 1, wherein the inner tube is able to move in an axial direction to the longitudinal axis by a plain bearing in the outer tube.

7. The leg prosthesis according to claim 1, wherein the outer tube and the inner tube are each configured as a tube slit in the direction of the longitudinal axis a or as a closed tube.

8. The leg prosthesis according to claim 1, wherein a guide pin is provided on the outer tube and an oblong hole on the inner tube, where the guide pin led in the oblong hole forms a tongue and groove connection, which secures the two tubes against twisting in the circumferential direction to the longitudinal axis a.

9. The leg prosthesis according to claim 1, wherein the retaining means or the guide pin or the outer tube are fashioned as end stop means that can bear against the inner tube in the position L or in the position K.

10. A system consisting of a leg prosthesis comprising: an outer tube having a longitudinal axis a and an inner tube able to move relative to the outer tube in the direction of the longitudinal axis, wherein a lockable retaining means is provided, by which the inner tube can be positively locked in the direction of the longitudinal axis a relative to the outer tube in at least one position L,
wherein a) the retaining means has a manually movable operating element, by which the retaining means is movable, so that one tube can be loosened in position L relative to the other tube and can be moved continuously by a force F into a position K, defined by an end stop with positive action in the direction of the longitudinal axis a, and
wherein b) the one tube starting from position K or any given position X between position K and position L can be moved automatically in the direction of the longitudinal axis a into position L solely by the force of gravity G acting on the tube, and in the exact position L the two tubes bear against each other by the positive end stop acting in the direction of the longitudinal axis a and are automatically secured by the retaining means, with a shaft arranged on the inner tube across an adapter and a foot fitting part arranged on the outer tube across an adapter.

11. The leg prosthesis according to claim 2, wherein the leg prosthesis can be shortened by a dimension M of at least 5 cm, starting from the position L, by the movement of the two tubes, and lengthened by the same dimension M of at least 5 cm, starting from the position K.

12. The leg prosthesis according to claim 11, wherein as the retaining means, a spring-loaded locking bolt is provided that can move in a radial direction to the longitudinal axis a, being mounted directly or indirectly on the outer tube and able to lock inside a borehole in the inner tube or in a hardened bushing in the inner tube.

13. The leg prosthesis according to claim 12, wherein the locking bolt is configured so that the locking in position L can be perceived acoustically and/or tactilely by a wearer of the leg prosthesis.

14. The leg prosthesis according to claim 13, wherein the inner tube is able to move in an axial direction to the longitudinal axis by a plain bearing in the outer tube.

15. The leg prosthesis according to claim 14, wherein the outer tube and the inner tube are each configured as a tube slit in the direction of the longitudinal axis a or as a closed tube.

16. The leg prosthesis according to claim 15, wherein a guide pin is provided on the outer tube and an oblong hole on the inner tube, where the guide pin led in the oblong hole forms a tongue and groove connection, which secures the two tubes against twisting in the circumferential direction to the longitudinal axis a.

17. The leg prosthesis according to claim 16, wherein the retaining means or the guide pin or the outer tube are fashioned as end stop means that can bear against the inner tube in the position L or in the position K.
